(19) United States
(12) Patent Application Publication
(10) Pub. No.: US 2025/0387407 A1
(43) Pub. Date: Dec. 25, 2025

(54) BIODEGRADABLE INTRANASAL SYSTEM FOR THE SUSTAINED-RELEASE OF AN ACTIVE INGREDIENT IN THE INTRANASAL CAVITY

(71) Applicants: DIANOSIC, STRASBOURG (FR); CENTRE NATIONAL DE LA RECHERCHE SCIENTIFIQUE, PARIS (FR); UNIVERSITE DE MONTPELLIER, MONTPELLIER (FR); ECOLE NATIONALE SUPERIEURE DE CHIMIE DE MONTPELLIER, MONTPELLIER CEDEX 5 (FR)

(72) Inventors: XAVIER GARRIC, MONTPELLIER (FR); SARAH ZAHOUANI, STRASBOURG (FR); VICTOR MOREAU, STRASBOURG (FR)

(21) Appl. No.: 18/881,381

(22) PCT Filed: Jul. 6, 2023
(86) PCT No.: PCT/EP2023/068784

§ 371 Date: Jan. 6, 2025

(30) Foreign Application Priority Data
Jul. 7, 2022 (EP) 22306018.7

(51) Int. Cl. A61K 31/56; A61K 9/00; A61K 47/34

(52) U.S. Cl. CPC A61K 31/56; A61K 9/0043; A61K 47/34

(57) ABSTRACT

The invention relates to biodegradable intranasal system for the sustained-release of fluticasone propionate in the intranasal cavity, said system comprising a biodegradable polyester matrix that comprises the fluticasone propionate as the active ingredient to be released in the intranasal cavity, wherein the polyester is selected from the list of poly(L,D-lactic acid) (PLA), poly(caprolactone) (PCL), their copolymers such as PLA-PCL, and mixtures thereof; and a method for preparing said system. The invention also relates to a kit comprising the system of the invention and the means of insertion of the system into the nasal cavity. The invention further relates to fluticasone propionate for use for treating chronic rhinitis or chronic sinusitis, wherein the fluticasone propionate is in a form suitable for administration in the nasal cavity by means of the biodegradable intranasal system of the invention.

The invention relates to a biodegradable intranasal system for the sustained-release of fluticasone propionate in the intranasal cavity

### TECHNOLOGICAL BACKGROUND

Chronic rhinitis is a long term inflammation of the inner lining of the nose, lasting for more than four consecutive weeks typically. Chronic rhinitis can be non-allergic or allergic rhinitis. Allergic rhinitis is caused by an allergic response to specific allergens, like pollen, dust, or pet dander. During an allergic response, your body's immune system is overreacting to the presence of one of these allergens in the air. Non-allergic rhinitis is any form of rhinitis that does not involve your body's immune system. It's often triggered by environmental issues, like air pollution, tobacco smoke, or strong odors. In some cases, a cause cannot be identified.

Chronic sinusitis is a long term sinus inflammation that typically lasts for more than 12 weeks, consecutive or not. Chronic sinusitis is different than recurrent sinusitis because chronic sinusitis symptoms never really go away for long periods of time. In recurrent sinusitis, one can have four or more bouts of sinusitis in one year, but may also have symptom-free periods in between.

Treatments of chronic rhinitis or chronic sinusitis usually involve a combination of medications such as antihistamines, antibiotics, saline nasal sprays, decongestants, corticosteroid, and/or anticholinergic. These medications include oral medications and nasal sprays. When medical treatment fails, energy based solutions such as laser, radiofrequency or cryotherapy may be proposed for chronic rhinitis. Surgery as surgery of the sinus in the case of chronic sinusitis or surgery of the inferior turbinate in the case of chronic rhinitis such as turbinoplasty or turbinectomy—the latter causing significant adverse events—may also be considered as an alternative

Fluticasone propionate is a corticosteroid usually used to relieve allergic or non-allergic nasal symptoms, such as stuffy/runny nose, itching, and sneezing. It works in the nose to block the effects of substances that cause allergies (such as pollen, pet dander, dust mites, mold) and to reduce swelling. In these applications, fluticasone propionate is administered locally by nasal sprays several times a day.

There is thus a need for easier administration and improved compliance of this medication. There is also a need for a sustained-release administration of this medication over a period of at least 6 months for chronic rhinitis or for at least 12 months for chronic sinusitis.

The international application WO 2006/107957 discloses a device for treating a paranasal sinus condition comprising a cavity member, a nasal portion and one or more active agent for sustained release into the sinus cavity. The device disclosed is configured to deliver an active agent for about one week to about one month (about 35 days).

The US patent application US 2013/281982 discloses an implantable device and a method for delivering a substance to a location within a paranasal sinus or nasal cavity, or an opening into a paranasal sinus or nasal cavity or a human or animal subject to treat a disorder such as sinusitis and other ear, nose and throat disorders.

However, there is a genuine need for devices that can be easily positioned in the nasal cavity and can release active agents to the intranasal cavity over an extended period of time of at least 3 months, preferably at least 6 months, typically for treating or preventing chronic rhinitis or chronic sinusitis.

### SUMMARY OF THE INVENTION

In this context, the inventors discovered a biodegradable intranasal system that can release the fluticasone propionate in the intranasal cavity during a period of at least 6 months, preferably between 6 and 12 months. In particular, the inventors discovered that a biodegradable system comprising a polyester matrix that comprises poly(L,D-lactic acid) (PDLLA), poly(D-lactic acid) (PDLA), poly(caprolactone) (PCL), copolymers of poly(caprolactone) (PCL) and poly(lactic acid) (PLA) such as PLA-PCL, or mixtures thereof, and the fluticasone propionate (FP), allows to release FP in the intranasal cavity during a period of at least 3 months, preferably at least 6 months, for example between 6 and 12 months. The inventors have shown that the degradation time of such a biodegradable system is more than 12 months, which means that there is no significant loss of mechanical properties during at least 12 months, thus ensuring a sufficient residence time in the intranasal cavity to release an effective amount of FP and thus treat chronic sinusitis or chronic rhinitis. Such a system is thus particularly suited for treating or preventing chronic sinusitis or chronic rhinitis. It has been shown that other polymers such as poly(L-lactic acid) (PLLA) or copolymer PLA-PEG-PLA cannot provide such a release or such a degradation time when taken alone.

The invention thus relates to a biodegradable intranasal system for the sustained-release of fluticasone propionate in the intranasal cavity, said system comprising a biodegradable polyester matrix that comprises the fluticasone propionate as the active ingredient to be released in the intranasal cavity, wherein the polyester is selected from the list of poly(L,D-lactic acid) (PLA), poly(caprolactone) (PCL), their copolymers such as PLA-PCL, and mixtures thereof.

The invention also relates to a kit comprising at least the system of the invention, and the means of insertion of said system into the nasal cavity.

The invention further relates to fluticasone propionate for use for treating chronic allergic or non-allergic rhinitis, wherein the fluticasone propionate is in a form suitable for administration in the nasal cavity by means of the biodegradable intranasal system of the invention.

The invention also relates to fluticasone propionate for use for treating chronic sinusitis, wherein the fluticasone propionate is in a form suitable for administration in the nasal cavity by means of the biodegradable intranasal system of the invention.

The invention further relates to a method for preparing the biodegradable intranasal system of the invention, said method comprising a step of forming the system by a treatment selected from extrusion, solvent evaporation (using for example dichloromethane), hot pressing, hot injection, freeze drying, electrospinning, molding or 3D printing.

### BRIEF DESCRIPTION OF THE DRAWINGS

FIG. 1 shows the evolution of the molar mass of a matrix PCL+10% FP as a function of time (see example 1). The samples were immersed in PBS pH 7.4, SDS 0.1% at 37° C. under stirring after TO.

FIG. 2 shows the evolution of the Young's modulus of a matrix PCL+10% FP as a function of time (see example 1). The samples were immersed in PBS pH 7.4, SDS 0.1% at 37° C. under stirring after TO.

FIG. 3 shows the evolution of the strain at break of a matrix PCL+10% FP as a function of time (see example 1). The samples were immersed in PBS pH 7.4, SDS 0.1% at 37° C. under stirring after TO.

FIG. 4 shows the release profiles of fluticasone propionate in PBS pH 7.4, SDS 0.1% from 7 types of films (PLA-PEG-PLA, PLA50, PLA85, PLA96, PLLA, PLA-PCL, PCL) formed by solvent evaporation—detection by HPLC: measurement of the area under the curve (AUC) of the absorbance peak of the molecule at 239 nm-retention time: 2.66 min (Kinetex® 2.6 μm C 18; mobile phase acetonitrile 60%, water 40%)-cumulative percentages.

FIG. 5 shows the release profiles of fluticasone propionate in PBS pH 7.4, SDS 0.1% from 4 types of polymer films (PLA-PEG-PLA, PLA50, PLLA and PCL) formed by hot press (“HP”) or extrusion and laser cutting. Detection by HPLC: measurement of the area under the curve (AUC) of the absorbance peak of the molecule at 239 nm-retention time: 2.66 min (Kinetex® 2.6 μm C 18; mobile phase acetonitrile 60%, water 40%)-cumulative percentages.

FIG. 6 shows the impact of gamma sterilization on the release profiles of fluticasone propionate in PBS pH 7.4, SDS 0.1% from a PCL film comprising 10% of FP. Detection by HPLC: measurement of the area under the curve (AUC) of the absorbance peak of the molecule at 239 nm-retention time: 2.66 min (Kinetex® 2.6 μm C 18; mobile phase acetonitrile 60%, water 40%)-cumulative percentages.

FIG. 7 shows the impact of thickness on the release profiles of fluticasone propionate in PBS pH 7.4, SDS 0.1% from a PCL film comprising 10% of FP. Detection by HPLC: measurement of the area under the curve (AUC) of the absorbance peak of the molecule at 239 nm-retention time: 2.66 min (Kinetex® 2.6 μm C 18; mobile phase acetonitrile 60%, water 40%)-cumulative percentages.

FIG. 8 shows the impact of FP percentage on the release profiles of fluticasone propionate in PBS pH 7.4, SDS 0.1% from a PCL film. Detection by HPLC: measurement of the air under the curve (AUC) of the absorbance peak of the molecule at 239 nm-retention time: 2.66 min (Kinetex® 2.6 μm C 18; mobile phase acetonitrile 60%, water 40%)-cumulative percentages.

FIG. 9 shows the release profiles of fluticasone propionate in PBS pH 7.4, SDS 0.1% from 11 types of polymer films (PLA-PCL copolymer, PCL, PLGA, 50/50 or 25/75 blends of PLA/PCL+PCL) formed by hot press (“HP”) or extrusion and laser cutting. Detection by HPLC: measurement of the area under the curve (AUC) of the absorbance peak of the molecule at 239 nm-retention time: 2.66 min (Kinetex® 2.6 μm C 18; mobile phase acetonitrile 60%, water 40%)-cumulative percentages.

FIG. 10 shows the release profiles of fluticasone propionate in PBS pH 7.4, SDS 0.5% from 11 types of polymer films (PLA-PCL copolymer, PCL, PLGA, 50/50 or 25/75 blends of PLA/PCL+PCL) formed by hot press (“HP”) or extrusion and laser cutting. Detection by HPLC: measurement of the area under the curve (AUC) of the absorbance peak of the molecule at 239 nm-retention time: 2.66 min (Kinetex® 2.6 μm C 18; mobile phase acetonitrile 60%, water 40%)-cumulative percentages.

### DETAILED DESCRIPTION

The inventors developed a biodegradable intranasal system for the sustained-release of fluticasone propionate in the intranasal cavity, having mechanical and chemical properties particularly suited to use in the medical field, and especially for treating or preventing chronic disorders of the nasal cavity such as chronic sinusitis or chronic rhinitis in the long term.

### Definition

In the context of the invention, the term “biodegradable system” refers to a system that is degradable in an aqueous or humid medium, in particular in biological medium such as in the intranasal cavity, in a specific or controlled period of time. The degradation leads to a loss in the mechanical properties of the system. In the context of the invention, the degradation of the system may be due to the progressive hydrolysis of the ester bonds in the polyester matrix. In a particular embodiment of the invention, the biodegradable system is resorbable.

In the context of the invention, the term “resorbable system” refers to a biodegradable system as defined above whose degradation products are metabolized.

According to the invention, an “aqueous medium” refers to a medium having a osmolarity similar to the osmolarity of biological fluids. Phosphate-buffered saline (PBS), regarded as representative of biological fluids, is commonly used as an aqueous medium.

According to the invention, a “humid medium” refers to a medium equivalent to the aqueous medium, i.e., a medium with an osmolarity similar to the osmolarity of biological fluids, but the humid medium is non-liquid and/or viscous. The intranasal cavity can be characterized as a non-liquid humid medium.

In the context of the invention, the term “intranasal cavity” or “nasal cavity” refers to a narrow space covered with mucous membrane that extends from the nostrils to the passageway to the pharynx (choana). It is divided into two cavities, also known as fossae (right and left) by a cartilage septum. On both sides of the nasal cavity are the turbinates: inferior, middle and superior turbinates.

In the context of the invention, the terms “molecular mass”, “molar mass” and “molecular weight” are used interchangeably to indicate, unless otherwise stated, the number average molecular/molar weight (Mn). According to the invention, Mn is determined by size-exclusion chromatography carried out in dimethylformamide as the analysis solvent, using a standard range of polystyrene.

In the context of the invention, the terms “biodegradable intranasal system of the invention”, “intranasal system of the invention” or “system of the invention” can be used interchangeably. Likewise, the terms “biodegradable polyester matrix” and “polyester matrix” can be used interchangeably

In the context of the invention, the term “about” means the given value plus or minus 10%.

In the context of the invention, the expression “between x and y” means that the values x and y are included.

### Biodegradable Intranasal System

The present invention relates to a biodegradable intranasal system for the sustained-release of fluticasone propionate in the intranasal cavity, said system comprising a biodegradable polyester matrix that comprises the fluticasone propionate as the active ingredient to be released in the intranasal cavity, wherein the polyester is selected from the list of poly(D,L-lactic acid) (PDLLA), poly(D-lactic acid) (PDLA), poly(caprolactone) (PCL), copolymers of poly(caprolactone) (PCL) and poly(lactic acid) (PLA) such as copolymers of PLA-PCL, and mixtures thereof.

The term “polyester” refers to a polymer whose repeat units of the main chain contain the ester function and which can be used in the medical field. In particular, “polyesters” means aliphatic polyesters such as poly(lactic acid), poly(glycolic acid), poly(caprolactone) (PCL), poly(butyrolactone) (PBL), poly(hydroxyalkanoates) (PHA), and copolymers thereof. In the context of the invention, the polyester is selected from poly(D,L-lactic acid) (PDLLA), poly(D-lactic acid) (PDLA), poly(caprolactone) (PCL) and copolymers of poly(caprolactone) (PCL) and poly(lactic acid) (PLA).

The poly(lactic acid) (PLA) can be poly(L-lactic acid) (PLLA), poly(D-lactic acid) (PDLA) or poly(D,L-lactic acid) (PDLLA). In the context of the invention, poly(D,L-lactic acid) and poly(D-lactic acid) are preferably used, in particular poly(D,L-lactic acid). In the case of poly(D,L-lactic acid) (PDLLA), the PLA comprises preferably at least 50% L-lactic acid, and in particular between 50% and 99% L-lactic acid. Typically, the poly(D,L-lactic acid) (PDLLA) can comprises 50% L-lactic acid (PLA50), 55% L-lactic acid (PLA55), 60% L-lactic acid (PLA60), 65% L-lactic acid (PLA65), 70% L-lactic acid (PLA70), 75% L-lactic acid (PLA75), 80% L-lactic acid (PLA80), 85% L-lactic acid (PLA85), 90% L-lactic acid (PLA90), 95% L-lactic acid (PLA95), 96% L-lactic acid (PLA96), or 99% L-lactic acid (PLA99). Preferably, the PDLLA comprises between 50% and 85% L-lactic acid, more preferably between 50% and 65% L-lactic acid. Indeed, by modifying the percentage of L-lactic acid in relation to D-lactic acid, it is possible to adjust the release of fluticasone propionate.

The copolymers of PLA and PCL can comprise PLA-PCL diblock, or PLA-PCL-PLA or PCL-PLA-PCL triblock copolymers, or statistical distribution of both monomers (in various proportions), or mixtures thereof, in particular [PLA-PCL-PLA and PCL-PLA-PCL], [PLA-PCL and or PCL-PLA-PCL], [PLA-PCL or PLA-PCL-PLA], [PLA-PCL-PLA and PCL-PLA-PCL and PCL]. Preferably, the system according to the invention comprises only PLA and PCL copolymer Preferably, the system according to the invention comprises only PLA and PCL block copolymer. In these copolymers, the PLA used is preferably PDLLA or PDLA, more preferably PDLLA. In a particular embodiment, the system according to the invention comprises only PLA-PCL diblock copolymer, and preferably only PLA85-PCL or PLA50-PCL diblock copolymers. In a particular embodiment, the PLA-PCL copolymer comprises a PLA/PCL molar ratio between 95/5 and 50/50. In a particular embodiment, the PLA-PCL diblock copolymer comprises a PLA/PCL molar ratio between 90/10 and 50/50, preferably between 90/10 and 70/30, more preferably of 85/15. The PLA/PCL molar ratio can be measured by proton nuclear magnetic resonance (1H NMR).

In a particular embodiment, the PLA-PCL copolymer comprises a PLA/PCL molar ratio between 95/5 and 50/50. In a particular embodiment, the PLA-PCL copolymer comprises a PLA/PCL molar ratio between 90/10 and 50/50, preferably between 90/10 and 70/30, more preferably of 85/15. The PLA/PCL molar ratio can be measured by proton nuclear magnetic resonance (1H NMR).

In a specific embodiment of the invention, the biodegradable polyester matrix can comprise a mixture of polyester selected from the list of PDLLA, PDLA, PCL, and copolymers of PCL and PLA such as copolymers PLA-PCL. Preferably, the biodegradable polyester matrix comprises a mixture of PCL and at least one polyester selected from the list of PDLLA, PDLA, and copolymers of PCL and PLA such as copolymers PLA-PCL. For example, the biodegradable polyester matrix can comprise a mixture of PCL, and a copolymer of PLA and PCL; preferably a mixture of PCL and a copolymer PLA-PCL.

In a specific embodiment, the biodegradable polyester matrix comprises a mixture of PCL and a copolymer PLA-PCL, preferably with a content of PLA-PCL from 0.1% to 60%, in relation to the total weight of the system. In this context, the content of PCL in the biodegradable polyester matrix is preferably at least 20% by weight, more preferably at least 40% by weight, in particular between 20% and 99.89% by weight, preferably between 40% and 99.89% by weight, in relation to the total weight of the system.

In a specific embodiment, the biodegradable polyester matrix comprises a mixture of PCL and a copolymer PLA-PCL in a ratio PCL/copolymer comprises between 10/90 and 90/10, preferably between 40/60 and 90/10, more preferably between 50/50 and 80/20, more preferably between 50/50 and 75/25. The inventors have observed that the more copolymer PLA-PCL the mixture contains, the slower the release of the fluticasone propionate.

In the context of the invention, when the biodegradable polyester matrix comprises PCL as the sole polyester, the content of PCL in the biodegradable polyester matrix is preferably at least 80% by weight, preferably at least 85% by weight, more preferably at least 90% by weight, in particular between 80% and 99.99% by weight, preferably between 85% and 99.99% by weight, more preferably between 90% and 99.99% by weight, in relation to the total weight of the system.

Preferably, the biodegradable polyester matrix comprises PCL or a mixture of PCL and PLA-PCL. More preferably, the biodegradable polyester matrix only comprises PCL as polyester.

In a preferred embodiment the PCL has a molar mass (in number) between 25 000 g/mol and 250 000 g/mol, preferably between 35 000 g/mol and 250 000 g/mol, preferably between 80 000 g/mol and 250 000 g/mol, preferably between 100 000 g/mol and 250 000 g/mol, more preferably between 120 000 g/mol and 250 000 g/mol, more preferably between 120 000 and 200 000 g/mol. The molar mass can be measured by means known by the one skilled in the art. For example, the average molar mass can be determined by size exclusion chromatography (SEC, Shimadzu SIL-20A HT) using two mixed medium columns PLgel 5 μm MIXED-C (300×7.8 mm), a Shimadzu RI detector 20-A and a Shimadzu UV detector SPD-20A (260 and 290 nm) (40° C. thermostatic analysis cells). DMF can be the mobile phase with 1 mL·min-1 flow at 40° C. (column temperature). The PCL can be dissolved in DMF to reach 5 mg·mL-1 concentration; afterwards, the solution can be filtered through a 0.45-μm Millipore filter before injection. Average molecular weights can be expressed according to calibration using polystyrene standards.

Preferably, the polyester of the matrix is in a non-crosslinked form.

In the context of the invention, the polyester matrix further comprises the fluticasone propionate. That means that the fluticasone propionate is dispersed in the polyester matrix. Preferably the fluticasone propionate is not covalently linked to the polyester in the polyester matrix.

Advantageously, the content of fluticasone propionate in the intranasal system according to the invention is at most 20% by weight, preferably at most 15% by weight, preferably at most 10% by weight, preferably between 0.01% and 15% by weight, preferably between 0.01% and 10% by weight, preferably between 0.1% and 10% by weight, more preferably between 1% and 10% by weight, based on the total weight of the system.

In a particular aspect of the invention, the polyester matrix consists of the polyester selected from the list of poly(D,L-lactic acid) (PDLLA), poly(D-lactic acid) (PDLA), poly(caprolactone) (PCL), copolymers of poly(caprolactone) (PCL) and poly(lactic acid) (PLA), and mixtures thereof; and the fluticasone propionate. Typically, the fluticasone propionate is incorporated into the structure of the polyester matrix comprising the polyester selected from the list of poly(D,L-lactic acid) (PDLLA), poly(D-lactic acid) (PDLA), poly(caprolactone) (PCL), copolymers of poly(caprolactone) (PCL) and poly(lactic acid) (PLA), and mixtures thereof.

In a particular aspect, the intranasal system of the invention is preferably made of the biodegradable polyester matrix. That means that the system of the invention preferably consists of the biodegradable polyester matrix. The system of the invention is thus preferably made of, or consists of, the polyester matrix comprising the fluticasone propionate and the polyester selected from the list of poly(D,L-lactic acid) (PDLLA), poly(D-lactic acid) (PDLA), poly(caprolactone) (PCL), copolymers of poly(caprolactone) (PCL) and poly(lactic acid) (PLA), and mixtures thereof.

In a particular embodiment, the invention thus relates to a composition comprising the polyester matrix according to the invention, as defined above. More particularly, the composition of the invention comprises, or consists of, the polyester matrix comprising the fluticasone propionate and the polyester selected from the list of poly(D,L-lactic acid) (PDLLA), poly(D-lactic acid) (PDLA), poly(caprolactone) (PCL), copolymers of poly(caprolactone) (PCL) and poly(lactic acid) (PLA), and mixtures thereof. More particularly, the composition of the invention comprises, or consists of, the polyester matrix comprising the fluticasone propionate and the polyester selected from the list of the poly(caprolactone) (PCL) and, a mixture of poly(caprolactone) (PCL) and PLA-PCL copolymer.

In a preferred embodiment, the intranasal system of the invention does not comprise a coating comprising an active ingredient such as the fluticasone propionate. In particular, the fluticasone propionate is only dispersed within the polyester matrix that constitutes the system of the invention. Preferably, the intranasal system of the invention does not comprise any coating.

In a specific embodiment, the polyester matrix consists of the fluticasone propionate and the polyester selected from the list of poly(D,L-lactic acid) (PDLLA), poly(D-lactic acid) (PDLA), poly(caprolactone) (PCL), copolymers of poly(caprolactone) (PCL) and poly(lactic acid) (PLA) such as PLA-PCL copolymers, and mixtures thereof.

In a specific embodiment, the polyester matrix consists of the fluticasone propionate and the polyester selected from the list of the poly(caprolactone) (PCL) and, a mixture of poly(caprolactone) (PCL) and PLA-PCL copolymer.

In another specific embodiment, the intranasal system of the invention can further comprise at least one homopolymer such as a polyester or a poly(ethylene glycol) (PEG). The homopolymer can be dispersed in the polyester matrix or can be in the form of a coating of the system. Preferably, the homopolymer is not covalently linked to the polyester matrix or the fluticasone propionate. This homopolymer can for example be added to the polyester matrix before or during the forming process, so as to be dispersed in the polyester matrix. Otherwise, it is possible to impregnate or coat the system or the polyester matrix with the homopolymer after the forming process. The addition of the homopolymer can provide a better control of the release of the fluticasone propionate, or of the mechanical properties of the system of the invention. Preferably, the homopolymer can be a poly(lactic acid) (PLA), poly(glycolic acid) (PGA), poly(caprolactone) (PCL), poly(butyrolactone) (PBL), poly(hydroxyalkanoates) (PHA), poly(ethylene glycol) (PEG), poly(oxyethylene) (PEO), or mixtures thereof. The homopolymer can have for example a molar mass (in number) of between 1 kDa and 300 kDa. Advantageously, the system of the invention comprises between 0.1% and 30% by weight, of the further homopolymer, in relation to the total weight of the system.

In another embodiment, the system of the invention can comprise, in addition to the polyester matrix, an additional additive or active ingredient, such as an antihistamine, another corticosteroid or anti-inflammatories. This additive or active ingredient can for example be added to polyester matrix before or during the forming of the matrix, so as to be dispersed in the polyester matrix. Otherwise, it is possible to impregnate or coat the system with this active ingredient or additive after the forming process. Preferably, the active ingredient is able to diffuse from the polymeric matrix/coating toward the external environment, when it is in an aqueous or humid medium.

In a specific aspect, the polyester matrix of the invention, and more particularly the intranasal system of the invention, has a Young's modulus comprised between 350 MPa and 1000 MPa, preferably between 350 MPa and 850 MPa, more preferably between 350 MPa and 650 MPa, even more preferably between 450 MPa and 650 MPa. The Young's modulus can be measured by any method known by the one skilled in the art. For example, the Young's modulus can be determined by performing a classic traction assay, and then by using OriginLab software to determine the values. In a specific aspect, when the polyester matrix comprises PCL as the sole polyester, the Young's modulus is comprised between 350 MPa and 1000 MPa, preferably between 350 MPa and 850 MPa, more preferably between 350 MPa and 650 MPa, even more preferably between 450 MPa and 650 MPa.

In a specific aspect, the polyester matrix of the invention, and more particularly the intranasal system of the invention, has a strain at break comprised between 100% and 700%, preferably between 300% and 500%, more preferably between 350% and 450%. The strain at break can be measured by any method known by the one skilled in the art. For example, the strain at break can be determined by performing a classic traction assay, and then by using OriginLab software to determine the values. In a specific aspect, when the polyester matrix comprises PCL as the sole polyester, the strain at break is comprised between 100% and 700%, preferably between 300% and 500%, more preferably between 350% and 450%.

In a specific aspect, the polyester matrix of the invention, and more particularly the intranasal system of the invention, can have a thickness between a few microns and several hundred microns, and particularly between 10 μm and 700 μm. Preferably, the intranasal system of the invention has a thickness between 100 μm and 700 μm, more preferably between 100 μm and 500 μm, such as between 100 μm and 400 μm. In a specific aspect, the polyester matrix of the invention, and more particularly the intranasal system of the invention, can have a thickness between 100 μm and 350 μm. Generally, the thickness of the system obtained depends on the quantity of the polyester matrix used and the surface area of the substrate or the mold used for the forming process. The thickness can be measured by methods commonly known in the art such as light microscopy. The control of the thickness of the system can provide a better control of the release of the fluticasone propionate. For example, the inventors have observed that the thicker the system, the slower the release of the fluticasone propionate.

The system according to the invention can thus take the form of a film, a tube, or other 2D or 3D matrix forms, etc. In particular, the system of the invention has a form adapted to the anatomy of the intranasal cavity, preferably adapted to be placed close to or around the inferior and/or medium turbinates.

A “film” means a two-dimensional material, for example resulting from the evaporation on a planar surface of the solvent having dissolved the polyester matrix according to the invention. The thickness of such a film is advantageously between a few microns and several hundred microns, and particularly between 10 μm and 700 μm. In a particular embodiment, the film has a thickness between about 100 μm and 700 μm, more preferably between 100 μm and 500 μm, such as between 100 μm and 400 μm. The thickness of such a film is advantageously between 100 μm and 350 μm. “Thickness” means “dry thickness”, in the sense that it is measured (for example by light microscopy) in anhydrous conditions, after forming and total evaporation of the solvent having dissolved the polyester matrix. The dimensions of the film can be adapted according to needs, in particular by cutting a film of greater dimensions to the desired dimensions.

The films can be folded to form tubes, or sleeves, held closed as needed by suturing or gluing. Tubes can also be obtained directly by being formed around a cylinder or by extrusion.

In the context of the invention, a “tube” refers to a hollow three-dimensional cylindrical object whose walls are formed of the polyester matrix according to the invention. Preferably, the diameter of such a tube is between 5 mm and 30 mm. In a particular embodiment, the tube has a wall thickness of between 100 μm and 700 μm and a diameter of between 5 mm and 30 mm.

In a specific aspect, the biodegradable intranasal system of the invention allows a sustained-release of the fluticasone propionate in the intranasal cavity during at least 90 days, preferably at least 105 days, preferably at least 120 days, preferably at least 135 days, preferably at least 150 days, preferably at least 165 days, preferably at least 180 days (i.e. 6 months). In a particular embodiment, the system of the invention can release the fluticasone propionate in the intranasal cavity during at least 180 days, preferably between 180 days and 365 days. In another particular embodiment, the system of the invention can release the fluticasone propionate in the intranasal cavity during at least 360 days, preferably between 360 days and 550 days. Advantageously, the sustained-release of the fluticasone propionate is a continuous sustained-release.

In a specific aspect, the biodegradable intranasal system of the invention can release at least 15% by weight, preferably at least 25% by weight, more preferably at least 40% by weight, of the fluticasone propionate initially present in the system after 180 days (i.e. after 6 months) after introduction of the system in an aqueous or humid medium. In a more specific aspect, the biodegradable intranasal system of the invention can release at least 50% by weight, preferably at least 70% by weight, more preferably at least 80% by weight, of the fluticasone propionate initially present in the system after 180 days (i.e. after 6 months) after introduction of the system in an aqueous or humid medium.

In another specific aspect, the biodegradable intranasal system of the invention can release at least 25% by weight, preferably at least 35% by weight, more preferably at least 45% by weight, of the fluticasone propionate initially present in the system after 360 days (i.e. after 12 months) after introduction of the system in an aqueous or humid medium. In a more specific aspect, the biodegradable intranasal system of the invention can release at least 50% by weight, preferably at least 70% by weight, more preferably at least 80% by weight, of the fluticasone propionate initially present in the system after 360 days (i.e. after 12 months) after introduction of the system in an aqueous or humid medium. In the context of the invention, the amounts of fluticasone propionate released at each release time can be measured by HPLC with a UV detector, fluorescence detector or mass spectrometer.

An additional particularly advantageous feature of the system according to the invention is that the system is degradable in an aqueous or humid medium (such as biological medium) after a residence time of at least 360 days, preferably at least 550 days. The loss of mechanical properties of the system of the invention is directly related to its degradation. Degradability can be evaluated for example by measuring the decrease in molecular weight of the system over time, after immersion at 37° C. in a saline medium (PBS 1X) under agitation, for example by Steric Exclusion Chromatography. The degradation of the system and so the solubilization of the polyester matrix in the intranasal cavity is gradual and controlled in order to allow the release of the fluticasone propionate in the intranasal cavity for a sufficient period of time before the mechanical properties of the system are impacted in a too large extent by degradation. The removal of the system of the invention can thus be a natural removal (after degradation or by resorption), or a removal by medical personnel. In a specific aspect of the invention, the intranasal system is resorbable.

### The Method for Preparing the Intranasal System of the Invention

The preparation of the intranasal system according to the invention can be done by any means known to the person skilled in the art, and in particular by incorporating the fluticasone propionate in the required content during the preparation of the polyester matrix.

The invention thus relates to a method for preparing the biodegradable intranasal system of the invention, said method comprising a step of forming the system by a treatment selected from extrusion, solvent evaporation (using for example dichloromethane), hot pressing, hot injection, freeze drying, electrospinning, molding or 3D printing, and advantageously a preliminary step of mixing together the polyester and the fluticasone propionate, before the treatment.

The preliminary step defined above can for example be prepared by incorporating the fluticasone propionate during the preparation of the polyester matrix by means commonly known by the one skilled in the art, including for example:
Impregnating/swelling the polyester of the polyester matrix in a solution comprising the fluticasone propionate;
Mixing dry powders of the polyester and the fluticasone propionate;
Mixing dry powders of the polyester and the fluticasone propionate in a liquid solvent (such as dichloromethane) in order to obtain a suspension or a solution;
Mixing by fusion or softening of powders of the polyester and the fluticasone propionate;
Mixing a solution of polyester and a powder of fluticasone propionate leading to a suspension or a solution; and
Mixing solutions of the polyester and the fluticasone propionate, optionally with a liquid solvent, leading to a suspension or a solution.

Preferably, the preliminary step comprises mixing dry powders or solutions of the polyester and the fluticasone propionate in a liquid solvent. The liquid solvent can for example be dichloromethane.

The system according to the invention can then be shaped by means known by the one skilled in the art and in particular by a treatment selected from extrusion, solvent evaporation (using for example dichloromethane), hot pressing, hot injection, freeze drying, electrospinning, molding or 3D printing, from a mixture comprising the polyester and the fluticasone propionate, in particular from the solution/suspension obtained during the preliminary step.

In a preferred embodiment, the intranasal system of the invention is prepared by mixing the polyester and the fluticasone propionate in a liquid solvent (such as dichloromethane) in order to obtain a suspension or a solution, and extruding the obtained suspension or solution while evaporating the liquid solvent.

In a specific embodiment, additional steps of cutting and/or assembling with means of intranasal insertion can be carried out.

After the manufacturing process, the system of the invention can be sterilized by means known by the one skilled in the art and for example by gamma sterilization, beta sterilization or ethylene oxide sterilization. In a preferred embodiment, the system of the invention is sterilized by gamma radiation, in particular by gamma radiation between 15 and 45 kGy.

### The Kit

The invention also relates to a kit comprising at least the biodegradable intranasal system of the invention and the means of insertion of the system into the nasal cavity.

The kit according to the invention, and in particular the means of insertion, enables the biodegradable intranasal system according to the invention to be reliably introduced and placed into the nasal cavity of a human body, preferably on a biological outgrowth present in the intranasal cavity, more preferably close to or around the inferior and/or medium turbinates.

The kit according to the invention can be used in particular in a subject suffering from chronic disorders of the nasal cavity, including chronic allergic or non-allergic rhinitis, and chronic sinusitis.

### The Drug Delivery System

The biodegradable intranasal system of the invention is particularly suitable for its use for treating or preventing chronic allergic or non-allergic rhinitis and/or chronic sinusitis, in a subject in need thereof.

In the context of the invention, the terms “subject”, “individual” or “patient” are interchangeable and refer to an animal, preferably to a mammal, even more preferably to a human. As defined herein, the subject suffers from chronic allergic or non-allergic rhinitis and/or chronic sinusitis. Said subject may be of any gender and any age.

According to the invention, the biodegradable intranasal system is preferably intended to be inserted into the intranasal cavity of a human body. The system of the invention is thus preferably suitable for being inserted and placed in a reliable manner into the intranasal cavity of a human body, preferably for being placed on a biological outgrowth present in the intranasal cavity, for example close to or around the inferior and/or medium turbinates.

The invention thus also relates to fluticasone propionate for use as an active ingredient for treating and/or preventing chronic disorders of the nasal cavity in a subject in need thereof, wherein the fluticasone propionate is in a form suitable for administration in the nasal cavity by means of a biodegradable intranasal system of the invention.

The invention thus also relates to fluticasone propionate for use as an active ingredient for treating and/or preventing chronic allergic or non-allergic rhinitis in a subject in need thereof, wherein the fluticasone propionate is in a form suitable for administration in the nasal cavity by means of a biodegradable intranasal system of the invention. In this embodiment, the biodegradable intranasal system of the invention is preferably suitable for releasing the fluticasone propionate in the nasal cavity during at least 120 days, preferably at least 180 days, in particular between 120 days and 240 days. Advantageously, the sustained-release of the fluticasone propionate is a continuous sustained-release.

The invention thus also relates to fluticasone propionate for use as an active ingredient for treating and/or preventing chronic sinusitis in a subject in need thereof, wherein the fluticasone propionate is in a form suitable for administration in the nasal cavity by means of a biodegradable intranasal system of the invention. In this embodiment, the biodegradable intranasal system of the invention is preferably suitable for releasing the fluticasone propionate in the nasal cavity during at least 240 days, preferably at least 360 days, in particular between 240 days and 550 days. Advantageously, the sustained-release of the fluticasone propionate is a continuous sustained-release.

The invention also relates to the composition of the invention for use for treating and/or preventing chronic disorders of the nasal cavity in a subject in need thereof, wherein the composition is in a form suitable for administration in the nasal cavity.

The invention thus also relates to the composition of the invention for use for treating and/or preventing chronic allergic or non-allergic rhinitis in a subject in need thereof, wherein the composition is in a form suitable for administration in the nasal cavity. In this embodiment, the composition of the invention is preferably suitable for releasing the fluticasone propionate in the nasal cavity during at least 120 days, preferably at least 180 days, in particular between 120 days and 240 days. Advantageously, the sustained-release of the fluticasone propionate is a continuous sustained-release.

The invention thus also relates to the composition of the invention for use for treating and/or preventing chronic sinusitis in a subject in need thereof, wherein the composition of the invention is in a form suitable for administration in the nasal cavity. In this embodiment, the composition of the invention is preferably suitable for releasing the fluticasone propionate in the nasal cavity during at least 240 days, preferably at least 360 days, in particular between 240 days and 550 days. Advantageously, the sustained-release of the fluticasone propionate is a continuous sustained-release.

The invention also relates to a method for treating and/or preventing chronic disorders of the nasal cavity in a subject in need thereof, comprising administering in the nasal cavity an effective amount of fluticasone propionate by means of a biodegradable intranasal system of the invention, in said subject.

The invention also relates to a method for treating and/or preventing chronic allergic or non-allergic rhinitis in a subject in need thereof, comprising administering in the nasal cavity an effective amount of fluticasone propionate by means of a biodegradable intranasal system of the invention, in said subject. In this embodiment, the biodegradable intranasal system of the invention is preferably suitable for releasing the fluticasone propionate in the nasal cavity during at least 120 days, preferably at least 180 days, in particular between 120 days and 240 days. Advantageously, the sustained-release of the fluticasone propionate is a continuous sustained-release.

The invention also relates to a method for treating and/or preventing chronic sinusitis in a subject in need thereof, comprising administering in the nasal cavity an effective amount of fluticasone propionate by means of a biodegradable intranasal system of the invention, in said subject. In this embodiment, the biodegradable intranasal system of the invention is preferably suitable for releasing the fluticasone propionate in the nasal cavity during at least 240 days, preferably at least 360 days, in particular between 240 days and 550 days. Advantageously, the sustained-release of the fluticasone propionate is a continuous sustained-release.

The invention also relates to a method for treating and/or preventing chronic disorders of the nasal cavity in a subject in need thereof, comprising administering a biodegradable intranasal system of the invention in the nasal cavity, in said subject.

The invention also relates to a method for treating and/or preventing chronic allergic or non-allergic rhinitis in a subject in need thereof, comprising administering a biodegradable intranasal system of the invention in the nasal cavity, in said subject. In this embodiment, the biodegradable intranasal system of the invention is preferably suitable for releasing the fluticasone propionate in the nasal cavity during at least 120 days, preferably at least 180 days, in particular between 120 days and 240 days. Advantageously, the sustained-release of the fluticasone propionate is a continuous sustained-release.

The invention also relates to a method for treating and/or preventing chronic sinusitis in a subject in need thereof, comprising administering a biodegradable intranasal system of the invention in the nasal cavity, in said subject. In this embodiment, the biodegradable intranasal system of the invention is preferably suitable for releasing the fluticasone propionate in the nasal cavity during at least 240 days, preferably at least 360 days, in particular between 240 days and 550 days. Advantageously, the sustained-release of the fluticasone propionate is a continuous sustained-release.

The invention also relates to the use of fluticasone propionate in a composition according to the invention, for the manufacture of a medicament for treating and/or preventing chronic disorders of the nasal cavity in a subject in need thereof, wherein the composition is in a form suitable for administration in the nasal cavity by means of a biodegradable intranasal system of the invention.

The invention also relates to the use of fluticasone propionate in a composition according to the invention, for the manufacture of a medicament for treating and/or preventing chronic allergic or non-allergic rhinitis in a subject in need thereof, wherein the composition is in a form suitable for administration in the nasal cavity by means of a biodegradable intranasal system of the invention. In this embodiment, the biodegradable intranasal system of the invention is preferably suitable for releasing the fluticasone propionate in the nasal cavity during at least 120 days, preferably at least 180 days, in particular between 120 days and 240 days. Advantageously, the sustained-release of the fluticasone propionate is a continuous sustained-release.

The invention also relates to the use of fluticasone propionate in a composition according to the invention, for the manufacture of a medicament for treating and/or preventing chronic sinusitis in need thereof, wherein the composition is in a form suitable for administration in the nasal cavity by means of a biodegradable intranasal system of the invention. In this embodiment, the biodegradable intranasal system of the invention is preferably suitable for releasing the fluticasone propionate in the nasal cavity during at least 240 days, preferably at least 360 days, in particular between 240 days and 550 days. Advantageously, the sustained-release of the fluticasone propionate is a continuous sustained-release.

The invention will now be illustrated using the examples below, which are presented for purposes of illustration and which in no way limit the invention.

### EXAMPLES

### Example 1: Preparation of Polyester Films Comprising Fluticasone Propionate

The polymers are:
poly(lactic acid) comprising 50% L-lactic acid (PLA 50): RESOMER® R 207 S-supplier Evonik (Mn≈185 000 g/mol);
poly(lactic acid) comprising 85% L-lactic acid (PLA85): RESOMER® LR 704 S-supplier Evonik (Mn≈262 000 g/mol);
poly(lactic acid) comprising 96% L-lactic acid (PLA96): PURASORB® PLD 9620-supplier Corbion (Mn≈ 136 800 g/mol);
poly(L-lactic acid) (PLLA): RESOMER® L 207 S-supplier Evonik (Mn≈141 000 g/mol supplier data);
poly(caprolactone) (PCL): molar mass (in number) of about 180 000 g/mol-RESOMER® C 217-supplier Evonik (Mn≈135 000 g/mol);
copolymer PLA-PEG-PLA: copolymer comprising 96% PLA85 and 4% poly(ethylene glycol) (PEG)-RESOMER® LRP t 7046-supplier Evonik (Mn≈158 000 g/mol); and
copolymer PLA-PCL: copolymer comprising 85% (w/w) PLA85 and 15% (w/w) PCL-PURASORB® PLC 8516-supplier Corbion (Mn≈149 000 g/mol).

Molar molecular weight have been determined according to the method described above.

The active ingredient is: Propionate fluticasone: CAS: 80474-14-2, Mw=500.58 g/mol During the experimental tests, glass materials are used such as glass pipette, glass vessel/container.

### 1-1) Preparation by Solvent Evaporation

Circular films containing 10% (w: w) of active ingredient (fluticasone propionate) were prepared by:
a) homogeneously mixing the fluticasone propionate with the polymer (PLA-PEG-PLA, PLA 50, PLA85, PLA96, PLA-PCL, PLLA, or PCL):
   1 g of fluticasone propionate is added to 9 g of polymer in a vial. 90 mL of analytical dichloromethane is added. The mixture is solubilized to obtain a solution;
b) forming a film by solvent evaporation:
   A precisely calculated volume of the solution is deposited on a substrate and the solvent is slowly evaporated to form a film; and then
c) cutting the film into 6 mm squares.

### 1-2) Preparation by Hot Press

Circular films containing 10% (w: w) of active ingredient (fluticasone propionate) were prepared by:
a) Homogeneously mixing the fluticasone propionate with the polymer (PLA-PEG-PLA, PLA 50, PLLA, or PCL:
   1 g of fluticasone propionate is added to 9 g of polymer in a vial. 90 mL of analytical dichloromethane is added. Once the mixture is solubilized, the dichloromethane is evaporated using a rotary evaporator under vacuum for 20 to 30 minutes. The vial is then placed under vacuum for 3 days (solvent trap immersed in liquid nitrogen connected between the vial and the vacuum pump) to remove residual solvent. Traces of residual solvent are considered to be removed when the mass of the vial reaches the mass it had initially. The resulting film is removed with a spatula, cut into very fine pieces with a chisel and placed at 4° C.;
b) Forming a film by hot pressing:
   A heat press, model Carver 41200E is used for this study. A Teflon paper mold (SS Shovan) is custom made in the laboratory to obtain circular films of 7 cm diameter with a thickness of 200 μm. Once the assembly is in place, a certain amount of the previously prepared film is placed in the center of the mold and then pressed. The parameters of temperature, amount of material, pressure and time under pressure are variable depending on the type of polymer chosen and are therefore optimized for each polymer under study (Table 1).
c) Laser cutting: the films thus obtained are laser cut to form sticks of 200 μm thickness with the following dimensions: 1.5 mm*24 mm. The thickness is measured with a micrometer (1866 HELIOS PREISSER);
d) Sterilizing by γ-radiation.

TABLE 1

| | | | Duration | Duration | Duration |
|---|---|---|---|---|---|
| | | Temperature | at 0 ton | at 1 ton | at 2 tons |
| | Mass (g) | (° C.) | (min) | (min) | (min) |
| PLA-PEG-PLA | 0.8-0.9 | 135-140 | 30 | 10 | 10-15 |
| PLA85 (L, D − | | | | | |
| 85:15) 4% PEG + | | | | | |
| 10% FP | | | | | |
| PLA 50 + 10% FP | 1-1.1 | 135 | 30 | 10 | 10 |
| PLLA + 10% FP | 0.8-0.98 | 190 | 30 | 10 | 0 |
| PCL + 10% FP | 0.8-1.05 | 100 | 30 | 10 | 10 |

### 1-3) Preparation by Extrusion

Tubes containing 10% (w: w) of active ingredient (fluticasone propionate) were prepared by:
a) homogeneously mixing the fluticasone propionate with the polymer (PLA-PEG-PLA, PLA 50, PLLA, or PCL:
   1 g of fluticasone propionate is added to 9 g of polymer in a vial. 90 mL of analytical dichloromethane is added. The mixture is solubilized to obtain a solution;
b) forming a film by extrusion:
   the solution is injected into the extruder (evaporation of the solvent during the extrusion) to produce tubes of 200 μm thickness (9 mm of internal diameter);
e) Laser cutting: the tubes thus obtained are laser cut to form sticks of 200 μm thickness with the following dimensions: 1.5 mm*24 mm. The thickness is measured with a micrometer (1866 HELIOS PREISSER) or calculated with the weight of the samples;
f) Sterilizing by γ-radiation.

### 1-4) Molar Mass Measurements

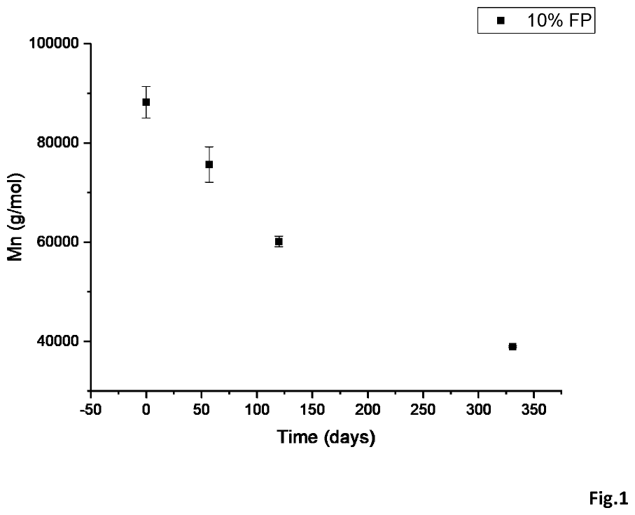

Average molar mass were determined by size exclusion chromatography (SEC, Shimadzu SIL-20A HT) using two mixed medium columns PLgel 5 μm MIXED-C (300×7.8 mm), a Shimadzu RI detector 20-A and a Shimadzu UV detector SPD-20A (260 and 290 nm) (40° C. thermostatic analysis cells). DMF was the mobile phase with 1 mL·min-1 Flow at 40° C. (column temperature). The polymer was dissolved in DMF to reach 5 mg·mL-1 concentration; afterwards, the solution was filtered through a 0.45-μm Millipore filter before injection. Average molecular weights were expressed according to calibration using polystyrene standards. FIG. 1 shows the evolution of the molar mass of the matrix PCL+10% FP as a function of time.

### 1-5) Characterization of Mechanical Properties

Samples of the matrix PCL+10% FP were used to investigate the evolution of mechanical properties during degradation. The shape of the samples (rectangular: 24 mm*1.5 mm) allowed to use them in traction assays. At defined times, samples were removed from release medium and dried using absorbent paper. For every sample (each time point was performed in triplicate):

When completely dried, the thickness of the sample was measured using a Helios-Preisser 1866-Basic 0-25 mm micrometer. The sample was positioned in an Instron 3344L8422 traction bench with 500 Newton jaws. The length of the sample between the two jaws was measured with a Helios-Preisser 2403 1850472 300 mm (12″) 0.01 mm (0.0005″) micrometer. A classic traction assay was performed at a speed of 10 mm/min. OriginLab software was used to calculate stress, strain and determine Young's modulus, stress and strain at break.

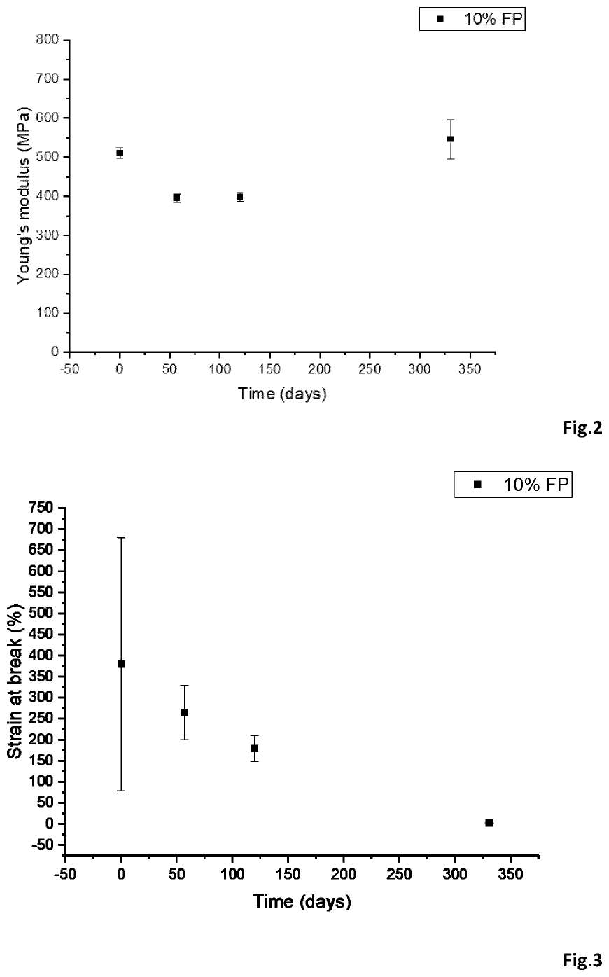

The evolution of Young's modulus as a function of time is shown in FIG. 2. The evolution of the strain at break as a function of time is shown in FIG. 3.

### Example 2: Release Profiles of Fluticasone Propionate from Films or Tubes Obtained in Example 1

Release medium: vials containing 20 mL of PBS (phosphate-buffered saline (pH 7.4)) containing 0.1% (w: v) SDS (sodium dodecyl sulfate) at 37° C. with agitation (100 rpm).

Sampling conditions: 1 mL of medium is taken and replaced by 1 mL of fresh medium during the first week of sampling, then the medium is completely renewed after each sampling of 1 mL every 7 days. The collected sample is then analyzed by HPLC.

2-1) Results of the Release of Fluticasone Propionate from the Films of Example 1-1):

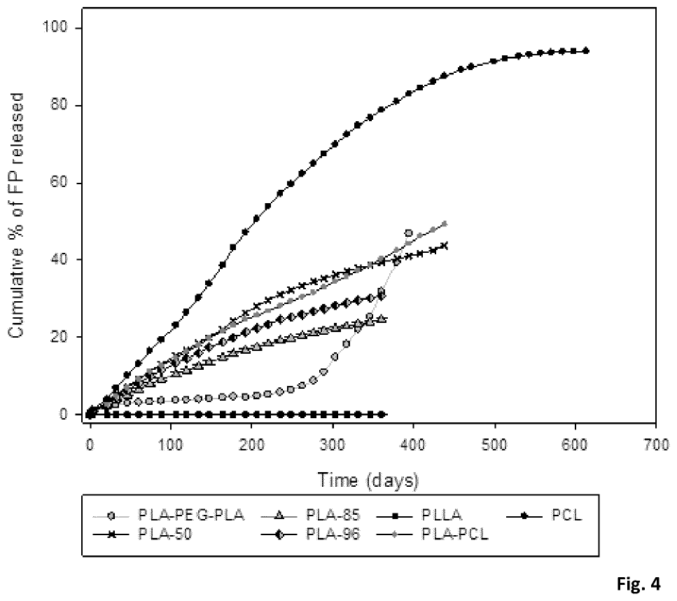

Each formulation (10% of fluticasone propionate w: w; 200 μm thickness) was tested in triplicate. The release profiles are shown in FIG. 4 (FIG. 4). The cumulative percentage release of FP increases over time for all films except for PLA-PEG-PLA films (the effect observed from 190 days onwards seems to be due to sample fragmentation) and PLLA films which do not seem to release the FP.

PLA50 and PLA-PCL films show the same release profiles. PLA85 and PLA96 films have release profiles close to each other and also close to those of PLA50 and PLA-PCL. PCL films release the most: about 80% of the total dose in about 12 months. It can be hypothesized that the fluticasone propionate has a higher affinity with the lactic acid monomer than with the caprolactone monomer.

These results confirm that a polyester matrix comprising fluticasone propionate and a polyester selected from PLA, PCL and copolymers thereof allows a sustained-release of the fluticasone propionate in an aqueous medium. These results also seem to confirm the greater affinity of the fluticasone propionate for lactic acid monomers than for caprolactone. A PCL matrix will thus be preferred for the invention.

2-2 Results of the Release of Fluticasone Propionate from the Films of Example 1-2) and 1-3):

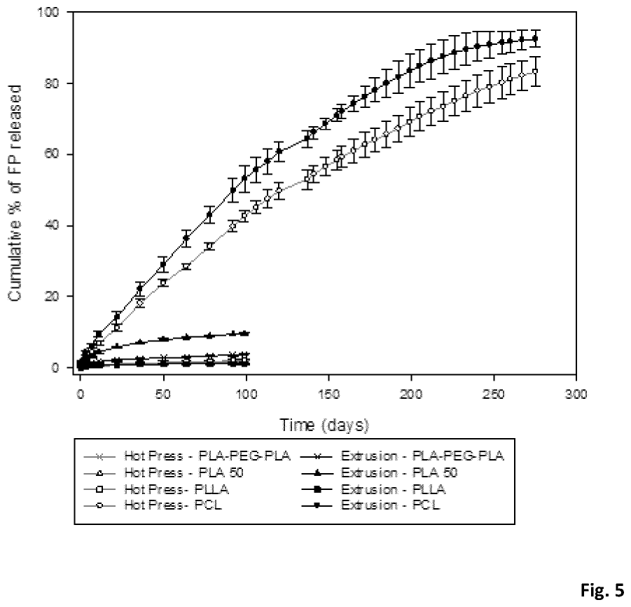

Each formulation (10% of fluticasone propionate w: w; 200 μm thickness) was tested in triplicate. The release profiles are shown in FIG. 5 (FIG. 5). It is noticeable that the formulations manufactured by extrusion seem to have a higher release kinetic the ones prepared by hot press. These results thus let suppose that the temperatures and mechanical constraints involved in the two manufacturing processes induce different mechanisms of repartition of the fluticasone propionate in the polymeric matrix.

2-3) Impact of Gamma Sterilization on the Release of Fluticasone Propionate from the PCL Films of Example 1-3);

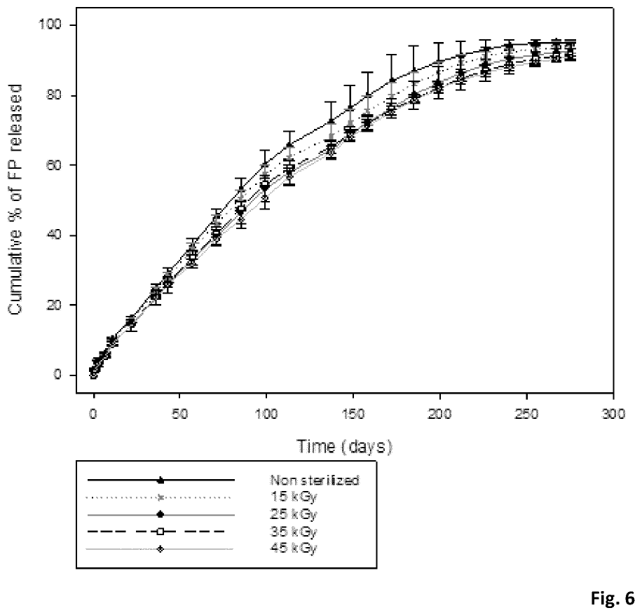

Each formulation (10% of fluticasone propionate w: w; 200 μm thickness) was tested in triplicate. The release profiles are shown in FIG. 6 (FIG. 6). It seems that the higher the gamma radiation dose is, the lower the release rate is. This could be a consequence of the reticulation of PCL chains at higher doses of gamma radiation. But this effect appears to be low. Gamma sterilization can thus be an appropriate sterilization process.

2-4) Impact of Thickness on the Release of Fluticasone Propionate from the PCL Films of Example 1.3:

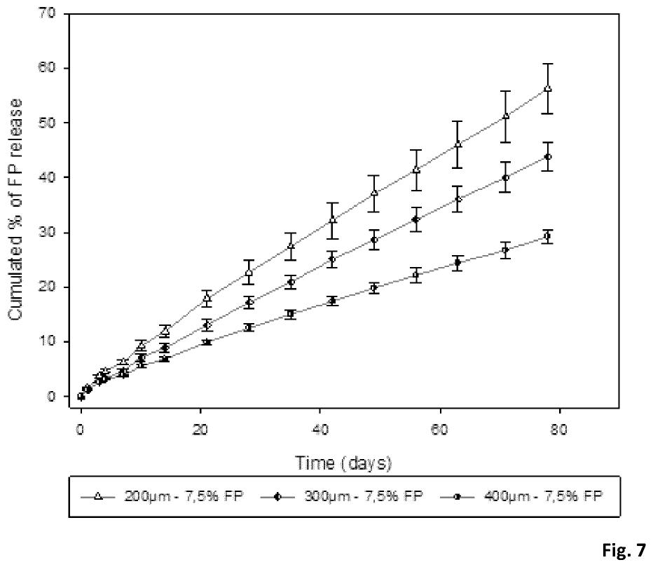

Each formulation (10% of fluticasone propionate w: w) was tested in triplicate. The release profiles are shown in FIG. 7 (FIG. 7). The results show that the higher the film thickness is, the slower the release is. It can thus be supposed that the mechanisms of diffusion and degradation involved in the release of the active molecule are slower with the increase of the thickness.

2-5) Impact of FP Percentage on the Release of Fluticasone Propionate from the PCL Films of Example 1.3:

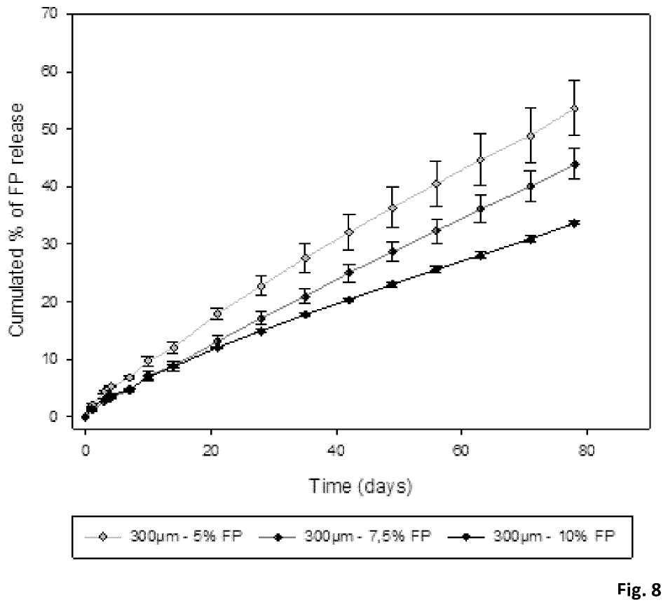

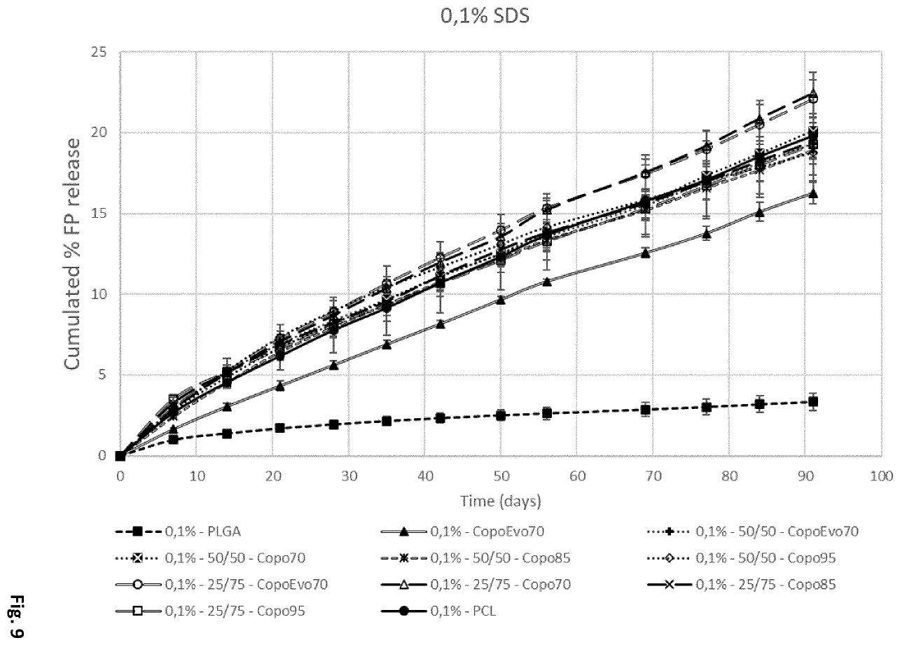

Each formulation (300 μm thickness) was tested in triplicate. The release profiles are shown in FIG. 8 (FIG. 8). The results show that the higher the FP percentage is, the slower the release is. It can thus be supposed as above that the mechanisms of diffusion and degradation involved in the release of the active molecule are slower with the increase of the percentage of FP.

### Example 3: Comparison of the Release Profiles of Fluticasone Propionate from Different Blends of PLA-PCL Copolymers and PCL Films and PLGA Tubes

### 3-1) Aim of the Study

This study reports the Fluticasone Propionate (FP) cumulative release profiles conducted on different blends of PLA-PCL copolymers and PCL films containing 10% of FP and on PLGA tubes containing 6% of FP.

### 3-2) Material and Methods

### Material

Ten formulations containing 10% of FP (w: w) were produced using polymers described in Table 2 below. Their average molecular weights are of same order of magnitude (around 100 kg/mol in number). With the exception of PCL (Resomer C 217) which is a linear homopolymer, polymers are linear statistical copolymers: the repartition of the monomers is random within each macromolecule.

TABLE 2

| | Commercial Reference | | | | |
|---|---|---|---|---|---|
| | PURASORB | RESOMER | PURASORB | PURASORB | RESOMER |
| | PLC 7015 | LC 703 S | PLC 8516 | PLC 9517 | C 217 |
| Identification | COPO70 | COPOEVO70 | COPO85 | COPO95 | PCL |

| | Chemical Structure | | | | |
|---|---|---|---|---|---|
| | co-PLA-PCL | co-PLA-PCL | co-PLA-PCL | co-PLA-PCL | |
| | 70% L-PLA/ | 70% L-PLA/ | 85% L-PLA/ | 95% L-PLA/ | 100% |
| | 30% PCL | 30% PCL | 15% PCL | 5% PCL | PCL |
| Batch | 7500065334 | D181200034 | 2010000753 | 1710002863 | D171200043 |
| number | | | | | |

PLGA tubes containing 6% of FP were cut with scissors and precisely weighted. These tubes could not be sterilized.

HPLC is performed with a Shimadzu model. The SPD-M20A IVDD photo diode array measures light absorbance of compounds eluted on a Kinetex® 2.6 μm C 18 100 Å LC column 100×46 mm. Release medium samples are analyzed following the “DEV-REC-Protocol for fluticasone propionate dosage by HPLC” parameters.

FP (CAS No.: 80474-14-2) is from Sterling, batch no FT-007/21, Phosphate-saline buffer (PBS) is from Sigma-Aldrich in tab form (P4417-100TAB), batch #SLCF6818 & SLCH0989, Sodium dodecyl sulfate (SDS) is from Sigma-Aldrich (436143-100G), batch n° #MKCJ9719.

HPLC grade acetonitrile, technical grade ethanol and purified water are used in this protocol. Solvents used as mobile phase (acetonitrile and water) contain 0.1% trifluoroacetic acid.

Glass vials of 20 ml volume are used to incubate the test items in release medium.

Glass vials of 1.5 ml volume are used for release medium samples analysis. A dedicated 1 ml glass graduated pipette is used to transfer release medium samples from incubation vials to analysis vials. A dedicated 10 ml glass graduated pipette is used to transfer fresh release medium into the incubation vials. This pipette never enters in contact with a solution containing FP.

Pipet-Aid XP from Drummond is used as a pipettor for all the sampling (1 and 10 ml pipettes).

Release samples are incubated in a heated shaker (Unimax 1010 & Inkubator 1000 models from Heidolph).

### Method

Release sample Preparation

In the following study, every condition is performed in triplicate on samples presented in Table 3 below.

TABLE 3

| | | | Corresponding | |
|---|---|---|---|---|
| | | FP | copolymer | PCL |
| | Sample | proportion | proportion | proportion |
| | reference | (w:w) | (w:w) | (w:w) |
| Controls | CopoEvo70 | 10% | 90% | 0% |
| | PCL | 10% | 0% | 90% |
| | PLGA | 6% | 94% | 0% |
| 50%-50% | 50-50 - CopoEvo70 | 10% | 45% | 45% |
| blends | 50-50 - Copo70 | 10% | 45% | 45% |
| | 50-50 - Copo85 | 10% | 45% | 45% |
| | 50-50 - Copo95 | 10% | 45% | 45% |
| 25%-75% | 25-75 - CopoEvo70 | 10% | 22.5% | 67.5% |
| blends | 25-75 - Copo70 | 10% | 22.5% | 67.5% |
| | 25-75 - Copo85 | 10% | 22.5% | 67.5% |
| | 25-75 - Copo95 | 10% | 22.5% | 67.5% |

330 μm mean thickness film are prepared by hot press method, as disclosed in example 1. The films were sterilized by gamma irradiation at 25 kGy by Ionisos and laser cut into 24 mm×1.5 mm sticks, then precisely weighted.

The 8 blend formulations, 2 “controls” (CopoEvo70 alone and PCL alone) and PLGA formulations were tested in two different release media: PBS, pH 7.4 supplemented with 0.1% SDS and PBS, pH 7.4 supplemented with 0.5% SDS.

Blanks in triplicate were used for each release medium (0.1% SDS and 0.5% SDS).

### Release Study Parameters

TABLE 4

| Release Medium | PBS supplemented with 0.1% SDS or | |
|---|---|---|
| | 0.5% SDS (w:v) (stored at room | |
| | temperature as SDS crystallizes at low temp) | |
| Release Volume | 20 ml per sample | |
| Frequency of release | Every 7 days | |
| medium change | | |
| Incubation temperature | 37° | C. |
| Incubation agitation | 100 | rpm |

| | |
|---|---|
| Sampling volume | 1 ml - After sampling, release |
| | medium is renewed. |
| Sampling frequency | every 7 days |

### 3-3) Results

### 0.1% SDS Release Medium

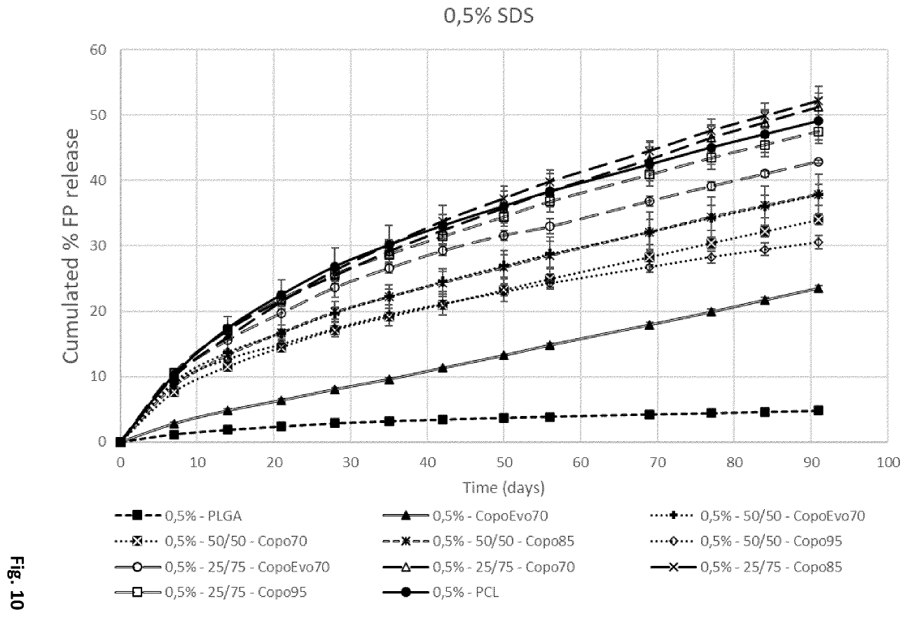

The results in FIG. 9 and Table 5 below show that after an initial burst, release of FP is low with a maximum of 3.3% released after 90 days when associated with PLGA polymer.

The formulation of pure PLA-PCL copolymer “CopoEvo70” shows a significantly slower release than PCL and the copolymer/PCL blends.

The copolymer/PCL blends and PCL alone present rather similar release profiles and their standard deviations are important.

On the last 40 days (From 50 to 90 days), 25% copolymer/75% PCL blends with Copo70 and CopoEvo70 tend to release more FP than PCL in 0.1% SDS, although standard deviations are still overlapping.

In the 0.1% SDS condition, the addition of a PLA-PCL copolymer with both studied ratios has little to no influence on the release profile of 10% FP loaded samples. The 0.5% SDS, considered as an accelerated condition, is more discriminant and is presented in the following section.

TABLE 5
Extracted data of cumulative percentages of fluticasone propionate
released in PBS, pH 7.4 supplemented with 0.1% SDS, n = 3

| | | | | 25%-75% blends | | | | 50%-50% blends | | | |
|---|---|---|---|---|---|---|---|---|---|---|---|
| Time | Controls | | | 25-75 - | 25-75 - | 25-75 - | 25-75 - | 50-50 - | 50-50 - | 50-50 - | 50-50 - |
| points | PLGA | CopoEvo70 | PCL | CopoEvo70 | Copo70 | Copo85 | Copo95 | CopoEvo70 | Copo70 | Copo85 | Copo95 |
| 14 days | 1% | 3% | 5% | 5% | 5% | 5% | 5% | 5% | 5% | 5% | 5% |
| 50 days | 2% | 10% | 12% | 14% | 14% | 14% | 12% | 12% | 12% | 12% | 13% |
| 91 days | 3% | 16% | 20% | 22% | 22% | 22% | 19% | 19% | 20% | 19% | 19% |

### 0.5% SDS Release Medium

The 0.5% SDS results presented in FIG. 10 show less overlapping release profiles than 0.1% SDS (FIG. 9). This tends to show that the 0.5% SDS condition is more discriminant for the release of the formulations evaluated in this study.

As expected, all formulations release greater proportions of FP quicker in 0.5% SDS than in 0.1% SDS. This is due to surfactant property of SDS, increasing FP solubility in water.

It is observed that the difference of SDS proportion in the release media have a low influence on PLGA formulation, the cumulative FP percentage released on day 50 is lower than 4% and lower than 5% after 90 days (see rounded results in tables 3 and 4), confirming the inadequacy of PLGA polymer with ARIS's specifications.

In this condition, the formulation containing pure PLA-PCL copolymer “CopoEvo70” (0.5%-CopoEvo70″) also presents a significantly slower release kinetic than other formulations. It can be observed that the difference of SDS proportion in the release media have a low influence on this formulation during the first two months: at day 50, 10% total FP was released in 0.1% SDS and 13% total FP was released in 0.5% SDS. But the gap between the two SDS conditions gets more significative after two months: 16% total FP was released at day 91 in 0.1% SDS and 24% total FP was released at day 91 in 0.5% SDS (data shown in tables 3 and 4). Comparing this formulation to PCL/PLA-PCL blends and PCL shows that formulating FP with the CopoEvo70 polymer reduces significantly FP's release kinetic.

Blends containing more copolymers (50-50 blends) present a slower release kinetic than PCL and the 25-75 blends. It shows that decreasing the PCL proportion reduces FP's release kinetic in PBS, pH 7.4+0.5% SDS.

After 50 days, release profiles of 25-75 blends of Copo95 and Copo70 are diverging. FP's release of Copo95 blend is decreasing comparing to all others.

FP release from pure CopoEvo70 formulation appears to be catching up the cumulative % of FP released of the 50-50 blends. It is observed that its release profile is more linear and does not exhibit a burst as important as PCL and all other formulations containing PCL (here, the burst corresponds to the important amount of FP released on the first 14 days).

### Visual Aspect of the Test Items

On the 7th day, all PLGA samples had importantly swollen and broken in half. This indicates an important water intake, which is not quantified as their FP release is still under evaluation. As a consequence, the samples have become more soft but maintain their integrity. All other samples did not show any visual change.

Until day 91, other samples did not visually change: neither fragmentation nor water intake were detected.

At 91 days of release, the following can be concluded:

PLGA is not a polyester suited for the sustained release of FP over time. Its release in FP is not sufficient and its important water intake makes it less adapted to a minimally invasive insert.

1-16. (canceled)

17. A biodegradable intranasal system for the sustained-release of fluticasone propionate in the intranasal cavity, said system comprising a biodegradable polyester matrix that comprises the fluticasone propionate as the active ingredient to be released in the intranasal cavity, wherein the polyester is selected from the group consisting of poly(D,L-lactic acid) (PDLLA), poly(D-lactic acid) (PDLA), poly(caprolactone) (PCL), copolymers of poly(caprolactone) (PCL) and poly(lactic acid) (PLA), and mixtures thereof.

18. The biodegradable intranasal system of claim 17, wherein the polyester is selected from the group consisting of a poly(caprolactone) (PCL), a poly(D,L-lactic acid) (PDLLA) comprising at least 50% L-lactic acid, PLA-PCL copolymers, and mixtures thereof.

19. The biodegradable intranasal system of claim 17, wherein the polyester is poly(caprolactone) (PCL) or a mixture of poly(caprolactone) (PCL) and PLA-PCL copolymer.

20. The biodegradable intranasal system of claim 17, wherein the polyester is a poly(caprolactone) (PCL).

21. The biodegradable intranasal system according to claim 17, wherein the poly(caprolactone) (PCL) has a molar mass between 25,000 g/mol and 250,000 g/mol.

22. The biodegradable intranasal system according to claim 17, wherein the content of fluticasone propionate is at most 20% by weight or is between 0.1% and 10% by weight, based on the total weight of the system.

23. The biodegradable intranasal system according to claim 17, wherein said system is made of the biodegradable polyester matrix that comprises the fluticasone propionate as the active ingredient to be released in the intranasal cavity.

24. The biodegradable intranasal system according to claim 17, wherein said system does not comprise a coating.

25. The biodegradable intranasal system according to claim 17, wherein at least 15% by weight of the fluticasone propionate initially present in the system is released after 180 days after introduction of the system in an aqueous or humid medium.

26. The biodegradable intranasal system according to claim 17, wherein said system releases the fluticasone propionate in the intranasal cavity for at least 120 days.

27. The biodegradable intranasal system according to claim 17, wherein said system releases the fluticasone propionate in the intranasal cavity for at least 240 days.

28. A kit comprising at least a system as defined in claim 17 and a means of insertion of the system into a nasal cavity.

29. A method of treating chronic allergic or non-allergic rhinitis comprising administering a biodegradable intranasal system as defined in claim 17 into the nasal cavity of a subject.

30. A method of treating chronic sinusitis comprising administering a biodegradable intranasal system as defined in claim 17 into the nasal cavity of a subject.

31. A method for preparing a biodegradable intranasal system comprising a step of forming the system by a treatment selected from extrusion, solvent evaporation, hot pressing, hot injection, freeze drying, electrospinning, molding or 3D printing, said system comprising a biodegradable polyester matrix that comprises fluticasone propionate as an active ingredient to be released in the intranasal cavity, wherein the polyester is selected from the group consisting of poly(D,L-lactic acid) (PDLLA), poly(D-lactic acid) (PDLA), poly(caprolactone) (PCL), copolymers of poly(caprolactone) (PCL) and poly(lactic acid) (PLA), and mixtures thereof.

32. The method of claim 31, comprising a preliminary step of mixing together the polyester and the fluticasone propionate, before the treatment step.
